(19) United States
(12) Patent Application Publication
(10) Pub. No.: US 2017/0238955 A1
(43) Pub. Date: Aug. 24, 2017

(54) Standing Tongue Cleaner

(71) Applicant: Spencer Cho, Lancaster, CA (US)

(72) Inventor: Spencer Cho, Lancaster, CA (US)

(21) Appl. No.: 15/052,619

(22) Filed: Feb. 24, 2016

(51) Int. Cl. A61B 17/24; A61B 17/32

(52) U.S. Cl. CPC A61B 17/244; A61B 17/320068; A61B 2017/320008

(57) ABSTRACT

A device with a removable and replaceable cleaning head for cleaning a surface. The cleaning head has a spoon shaped head section configured to be operable to reduce a user's gag reflex. A cleaning blade segment is configured to form a triple edged surface scraping implement and a ball base segment is configured to aid a user in the installation and removal of said cleaning head component. A base component is configured to attach to said cleaning head component including a motor that is configured to vibrate said cleaning head component at a predetermined rate of a direction of motion of said cleaning head. An indicator indicates a status of said device.

### CROSS-REFERENCE TO RELATED APPLICATIONS

Not applicable.

### RELATED CO-PENDING U.S. PATENT APPLICATIONS

Not applicable.

### FEDERALLY SPONSORED RESEARCH OR DEVELOPMENT

Not applicable.

### REFERENCE TO SEQUENCE LISTING, A TABLE, OR A COMPUTER LISTING APPENDIX

Not applicable.

### COPYRIGHT NOTICE

A portion of the disclosure of this patent document contains material that is subject to copyright protection by the author thereof. The copyright owner has no objection to the facsimile reproduction by anyone of the patent document or patent disclosure for the purposes of referencing as patent prior art, as it appears in the Patent and Trademark Office, patent file or records, but otherwise reserves all copyright rights whatsoever.

### FIELD OF THE INVENTION

One or more embodiments of the invention generally relate to oral cleaners. More particularly, the invention relates to power tongue cleaners.

### BACKGROUND OF THE INVENTION

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

Tongue scraping is an oral hygiene technique that removes residue, bacteria, toxins, and/or other debris from a user's tongue. The presence of residue, bacteria, and/or toxins on a user's tongue may contribute to bad breath, a weakened immune system, poor digestion, and/or poor dental health. Typically, bacteria and toxins are located in the deep crevices of a person's tongue and may be difficult to remove by brushing and/or flossing.

A tongue scraper is a tool that may assist a user in removing residue and/or bacteria from the user's tongue. A typical tongue scraper is a mechanical device with a blade that is used to physically scrape residue from the surface of a user's tongue. Current tongue scrapers may also vibrate at a certain frequency to assist in the removal of residue on a user's tongue.

The following is an example of a specific aspect in the prior art that, while expected to be helpful to further educate the reader as to additional aspects of the prior art, is not to be construed as limiting the present invention, or any embodiments thereof, to anything stated or implied therein or inferred thereupon. By way of educational background, another aspect of the prior art generally useful to be aware of is that some existing tongue scrapers may be designed in a shape that ineffectively removes residue and/or bacteria from a user's tongue. Other designs may not be structurally durable and may break during use.

In view of the foregoing, it is clear that these traditional techniques are not perfect and leave room for more optimal approaches.

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1 illustrates an outside view of an exemplary power tongue cleaner, in accordance with an embodiment of the present invention;

FIG. 2 illustrates an internal view of an exemplary power tongue cleaner, in accordance with an embodiment of the present invention;

FIG. 3 illustrates an outside view of an exemplary cleaning head, in accordance with an embodiment of the present invention;

FIG. 4 illustrates an internal view of an exemplary cleaning head, in accordance with an embodiment of the present invention.

Unless otherwise indicated illustrations in the figures are not necessarily drawn to scale.

### DETAILED DESCRIPTION OF SOME EMBODIMENTS

The present invention is best understood by reference to the detailed figures and description set forth herein.

Embodiments of the invention are discussed below with reference to the Figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

It is to be further understood that the present invention is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub-steps and subservient means. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

All words of approximation as used in the present disclosure and claims should be construed to mean “approximate,” rather than “perfect,” and may accordingly be employed as a meaningful modifier to any other word, specified parameter, quantity, quality, or concept. Words of approximation, include, yet are not limited to terms such as “substantial”, “nearly”, “almost”, “about”, “generally”, “largely”, “essentially”, “closely approximate”, etc.

As will be established in some detail below, it is well settle law, as early as 1939, that words of approximation are not indefinite in the claims even when such limits are not defined or specified in the specification.

For example, see Ex parte Mallory, 52 USPQ 297, 297 (Pat. Off. Bd. App. 1941) where the court said “The examiner has held that most of the claims are inaccurate because apparently the laminar film will not be entirely eliminated. The claims specify that the film is “substantially” eliminated and for the intended purpose, it is believed that the slight portion of the film which may remain is negligible. We are of the view, therefore, that the claims may be regarded as sufficiently accurate.”

Note that claims need only “reasonably apprise those skilled in the art” as to their scope to satisfy the definiteness requirement. See Energy Absorption Sys., Inc. v. Roadway Safety Servs., Inc., Civ. App. 96-1264, slip op. at 10 (Fed. Cir. Jul. 3, 1997) (unpublished) Hybridtech v. Monoclonal Antibodies, Inc., 802 F.2d 1367, 1385, 231 USPQ 81, 94 (Fed. Cir. 1986), cert. denied, 480 U.S. 947 (1987). In addition, the use of modifiers in the claim, like “generally” and “substantial,” does not by itself render the claims indefinite. See Seattle Box Co. v. Industrial Crating & Packing, Inc., 731 F.2d 818, 828-29, 221 USPQ 568, 575-76 (Fed. Cir. 1984).

Moreover, the ordinary and customary meaning of terms like “substantially” includes “reasonably close to: nearly, almost, about”, connoting a term of approximation. See In re Frye, Appeal No. 2009-006013, 94 USPQ2d 1072, 1077, 2010 WL 889747 (B.P.A.I. 2010) Depending on its usage, the word “substantially” can denote either language of approximation or language of magnitude. Deering Precision Instruments, L.L.C. v. Vector Distribution Sys., Inc., 347 F.3d 1314, 1323 (Fed. Cir. 2003) (recognizing the “dual ordinary meaning of th[e] term [“substantially”] as connoting a term of approximation or a term of magnitude”). Here, when referring to the “substantially halfway” limitation, the Specification uses the word “approximately” as a substitute for the word “substantially” (Fact 4). (Fact 4). The ordinary meaning of “substantially halfway” is thus reasonably close to or nearly at the midpoint between the forwardmost point of the upper or outsole and the rearwardmost point of the upper or outsole.

Similarly, the term ‘substantially’ is well recognize in case law to have the dual ordinary meaning of connoting a term of approximation or a term of magnitude. See Dana Corp. v. American Axle & Manufacturing, Inc., Civ. App. 04-1116, 2004 U.S. App. LEXIS 18265, *13-14 (Fed. Cir. Aug. 27, 2004) (unpublished). The term “substantially” is commonly used by claim drafters to indicate approximation. See Cordis Corp. v. Medtronic AVE Inc., 339 F.3d 1352, 1360 (Fed. Cir. 2003) (“The patents do not set out any numerical standard by which to determine whether the thickness of the wall surface is ‘substantially uniform.’ The term ‘substantially,’ as used in this context, denotes approximation. Thus, the walls must be of largely or approximately uniform thickness.”); see also Deering Precision Instruments, LLC v. Vector Distribution Sys., Inc., 347 F.3d 1314, 1322 (Fed. Cir. 2003); Epcon Gas Sys., Inc. v. Bauer Compressors, Inc., 279 F.3d 1022, 1031 (Fed. Cir. 2002). We find that the term “substantially” was used in just such a manner in the claims of the patents-in-suit: “substantially uniform wall thickness” denotes a wall thickness with approximate uniformity.

It should also be noted that such words of approximation as contemplated in the foregoing clearly limits the scope of claims such as saying ‘generally parallel’ such that the adverb ‘generally’ does not broaden the meaning of parallel. Accordingly, it is well settled that such words of approximation as contemplated in the foregoing (e.g., like the phrase ‘generally parallel’) envisions some amount of deviation from perfection (e.g., not exactly parallel), and that such words of approximation as contemplated in the foregoing are descriptive terms commonly used in patent claims to avoid a strict numerical boundary to the specified parameter. To the extent that the plain language of the claims relying on such words of approximation as contemplated in the foregoing are clear and uncontradicted by anything in the written description herein or the figures thereof, it is improper to rely upon the present written description, the figures, or the prosecution history to add limitations to any of the claim of the present invention with respect to such words of approximation as contemplated in the foregoing. That is, under such circumstances, relying on the written description and prosecution history to reject the ordinary and customary meanings of the words themselves is impermissible. See, for example, Liquid Dynamics Corp. v. Vaughan Co., 355 F.3d 1361, 69 USPQ2d 1595, 1600-01 (Fed. Cir. 2004). The plain language of phrase 2 requires a “substantial helical flow.” The term “substantial” is a meaningful modifier implying “approximate,” rather than “perfect.” In Cordis Corp. v. Medtronic AVE, Inc., 339 F.3d 1352, 1361 (Fed. Cir. 2003), the district court imposed a precise numeric constraint on the term “substantially uniform thickness.” We noted that the proper interpretation of this term was “of largely or approximately uniform thickness” unless something in the prosecution history imposed the “clear and unmistakable disclaimer” needed for narrowing beyond this simple-language interpretation. Id. In Anchor Wall Systems v. Rockwood Retaining Walls, Inc., 340 F.3d 1298, 1311 (Fed. Cir. 2003)” Id. at 1311. Similarly, the plain language of claim 1 requires neither a perfectly helical flow nor a flow that returns precisely to the center after one rotation (a limitation that arises only as a logical consequence of requiring a perfectly helical flow).

The reader should appreciate that case law generally recognizes a dual ordinary meaning of such words of approximation, as contemplated in the foregoing, as connoting a term of approximation or a term of magnitude; e.g., see Deering Precision Instruments, L.L.C. v. Vector Distrib. Sys., Inc., 347 F.3d 1314, 68 USPQ2d 1716, 1721 (Fed. Cir. 2003), cert. denied, 124 S. Ct. 1426 (2004) where the court was asked to construe the meaning of the term “substantially” in a patent claim. Also see Epcon, 279 F.3d at 1031 (“The phrase ‘substantially constant’ denotes language of approximation, while the phrase ‘substantially below’ signifies language of magnitude, i.e., not insubstantial.”). Also, see, e.g., Epcon Gas Sys., Inc. v. Bauer Compressors, Inc., 279 F.3d 1022 (Fed. Cir. 2002) (construing the terms “substantially constant” and “substantially below”); Zodiac Pool Care, Inc. v. Hoffinger Indus., Inc., 206 F.3d 1408 (Fed. Cir. 2000) (construing the term “substantially inward”); York Prods., Inc. v. Cent. Tractor Farm & Family Ctr., 99 F.3d 1568 (Fed. Cir. 1996) (construing the term “substantially the entire height thereof”); Tex. Instruments Inc. v. Cypress Semiconductor Corp., 90 F.3d 1558 (Fed. Cir. 1996) (construing the term “substantially in the common plane”). In conducting their analysis, the court instructed to begin with the ordinary meaning of the claim terms to one of ordinary skill in the art. Prima Tek, 318 F.3d at 1148. Reference to dictionaries and our cases indicates that the term “substantially” has numerous ordinary meanings. As the district court stated, “substantially” can mean “significantly” or “considerably.” The term “substantially” can also mean “largely” or “essentially.” Webster's New 20th Century Dictionary 1817 (1983).

Words of approximation, as contemplated in the foregoing, may also be used in phrases establishing approximate ranges or limits, where the end points are inclusive and approximate, not perfect; e.g., see AK Steel Corp. v. Sollac, 344 F.3d 1234, 68 USPQ2d 1280, 1285 (Fed. Cir. 2003) where it where the court said [W]e conclude that the ordinary meaning of the phrase “up to about 10%” includes the “about 10%” endpoint. As pointed out by AK Steel, when an object of the preposition “up to” is nonnumeric, the most natural meaning is to exclude the object (e.g., painting the wall up to the door). On the other hand, as pointed out by Sollac, when the object is a numerical limit, the normal meaning is to include that upper numerical limit (e.g., counting up to ten, seating capacity for up to seven passengers). Because we have here a numerical limit—“about 10%”—the ordinary meaning is that that endpoint is included.

In the present specification and claims, a goal of employment of such words of approximation, as contemplated in the foregoing, is to avoid a strict numerical boundary to the modified specified parameter, as sanctioned by Pall Corp. v. Micron Separations, Inc., 66 F.3d 1211, 1217, 36 USPQ2d 1225, 1229 (Fed. Cir. 1995) where it states “It is well established that when the term “substantially” serves reasonably to describe the subject matter so that its scope would be understood by persons in the field of the invention, and to distinguish the claimed subject matter from the prior art, it is not indefinite.” Likewise see Verve LLC v. Crane Cams Inc., 311 F.3d 1116, 65 USPQ2d 1051, 1054 (Fed. Cir. 2002). Expressions such as “substantially” are used in patent documents when warranted by the nature of the invention, in order to accommodate the minor variations that may be appropriate to secure the invention. Such usage may well satisfy the charge to “particularly point out and distinctly claim” the invention, 35 U.S.C. §112, and indeed may be necessary in order to provide the inventor with the benefit of his invention. In Andrew Corp. v. Gabriel Elecs. Inc., 847 F.2d 819, 821-22, 6 USPQ2d 2010, 2013 (Fed. Cir. 1988) the court explained that usages such as “substantially equal” and “closely approximate” may serve to describe the invention with precision appropriate to the technology and without intruding on the prior art. The court again explained in Ecolab Inc. v. Envirochem, Inc., 264 F.3d 1358, 1367, 60 USPQ2d 1173, 1179 (Fed. Cir. 2001) that “like the term ‘about,’ the term ‘substantially’ is a descriptive term commonly used in patent claims to ‘avoid a strict numerical boundary to the specified parameter, see Ecolab Inc. v. Envirochem Inc., 264 F.3d 1358, 60 USPQ2d 1173, 1179 (Fed. Cir. 2001) where the court found that the use of the term “substantially” to modify the term “uniform” does not render this phrase so unclear such that there is no means by which to ascertain the claim scope.

Similarly, other courts have noted that like the term “about,” the term “substantially” is a descriptive term commonly used in patent claims to “avoid a strict numerical boundary to the specified parameter.”; e.g., see Pall Corp. v. Micron Seps., 66 F.3d 1211, 1217, 36 USPQ2d 1225, 1229 (Fed. Cir. 1995); see, e.g., Andrew Corp. v. Gabriel Elecs. Inc., 847 F.2d 819, 821-22, 6 USPQ2d 2010, 2013 (Fed. Cir. 1988) (noting that terms such as “approach each other,” “close to,” “substantially equal,” and “closely approximate” are ubiquitously used in patent claims and that such usages, when serving reasonably to describe the claimed subject matter to those of skill in the field of the invention, and to distinguish the claimed subject matter from the prior art, have been accepted in patent examination and upheld by the courts). In this case, “substantially” avoids the strict 100% nonuniformity boundary.

Indeed, the foregoing sanctioning of such words of approximation, as contemplated in the foregoing, has been established as early as 1939, see Ex parte Mallory, 52 USPQ 297, 297 (Pat. Off. Bd. App. 1941) where, for example, the court said “the claims specify that the film is “substantially” eliminated and for the intended purpose, it is believed that the slight portion of the film which may remain is negligible. We are of the view, therefore, that the claims may be regarded as sufficiently accurate.” Similarly, In re Hutchison, 104 F.2d 829, 42 USPQ 90, 93 (C.C.P.A. 1939) the court said “It is realized that “substantial distance” is a relative and somewhat indefinite term, or phrase, but terms and phrases of this character are not uncommon in patents in cases where, according to the art involved, the meaning can be determined with reasonable clearness.”

Hence, for at least the forgoing reason, Applicants submit that it is improper for any examiner to hold as indefinite any claims of the present patent that employ any words of approximation.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention. Structures described herein are to be understood also to refer to functional equivalents of such structures. The present invention will now be described in detail with reference to embodiments thereof as illustrated in the accompanying drawings.

From reading the present disclosure, other variations and modifications will be apparent to persons skilled in the art. Such variations and modifications may involve equivalent and other features which are already known in the art, and which may be used instead of or in addition to features already described herein.

Although Claims have been formulated in this Application to particular combinations of features, it should be understood that the scope of the disclosure of the present invention also includes any novel feature or any novel combination of features disclosed herein either explicitly or implicitly or any generalization thereof, whether or not it relates to the same invention as presently claimed in any Claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable subcombination. The Applicants hereby give notice that new Claims may be formulated to such features and/or combinations of such features during the prosecution of the present Application or of any further Application derived therefrom.

References to “one embodiment,” “an embodiment,” “example embodiment,” “various embodiments,” “some embodiments,” “embodiments of the invention,” etc., may indicate that the embodiment(s) of the invention so described may include a particular feature, structure, or characteristic, but not every possible embodiment of the invention necessarily includes the particular feature, structure, or characteristic. Further, repeated use of the phrase “in one embodiment,” or “in an exemplary embodiment,” “an embodiment,” do not necessarily refer to the same embodiment, although they may. Moreover, any use of phrases like “embodiments” in connection with “the invention” are never meant to characterize that all embodiments of the invention must include the particular feature, structure, or characteristic, and should instead be understood to mean “at least some embodiments of the invention” includes the stated particular feature, structure, or characteristic.

References to “user”, or any similar term, as used herein, may mean a human or non-human user thereof. Moreover, “user”, or any similar term, as used herein, unless expressly stipulated otherwise, is contemplated to mean users at any stage of the usage process, to include, without limitation, direct user(s), intermediate user(s), indirect user(s), and end user(s). The meaning of “user”, or any similar term, as used herein, should not be otherwise inferred or induced by any pattern(s) of description, embodiments, examples, or referenced prior-art that may (or may not) be provided in the present patent.

References to “end user”, or any similar term, as used herein, is generally intended to mean late stage user(s) as opposed to early stage user(s). Hence, it is contemplated that there may be a multiplicity of different types of “end user” near the end stage of the usage process. Where applicable, especially with respect to distribution channels of embodiments of the invention comprising consumed retail products/services thereof (as opposed to sellers/vendors or Original Equipment Manufacturers), examples of an “end user” may include, without limitation, a “consumer”, “buyer”, “customer”, “purchaser”, “shopper”, “enjoyer”, “viewer”, or individual person or non-human thing benefiting in any way, directly or indirectly, from use of. or interaction, with some aspect of the present invention.

In some situations, some embodiments of the present invention may provide beneficial usage to more than one stage or type of usage in the foregoing usage process. In such cases where multiple embodiments targeting various stages of the usage process are described, references to “end user”, or any similar term, as used therein, are generally intended to not include the user that is the furthest removed, in the foregoing usage process, from the final user therein of an embodiment of the present invention.

Where applicable, especially with respect to retail distribution channels of embodiments of the invention, intermediate user(s) may include, without limitation, any individual person or non-human thing benefiting in any way, directly or indirectly, from use of, or interaction with, some aspect of the present invention with respect to selling, vending, Original Equipment Manufacturing, marketing, merchandising, distributing, service providing, and the like thereof.

References to “person”, “individual”, “human”, “a party”, “animal”, “creature”, or any similar term, as used herein, even if the context or particular embodiment implies living user, maker, or participant, it should be understood that such characterizations are sole by way of example, and not limitation, in that it is contemplated that any such usage, making, or participation by a living entity in connection with making, using, and/or participating, in any way, with embodiments of the present invention may be substituted by such similar performed by a suitably configured non-living entity, to include, without limitation, automated machines, robots, humanoids, computational systems, information processing systems, artificially intelligent systems, and the like. It is further contemplated that those skilled in the art will readily recognize the practical situations where such living makers, users, and/or participants with embodiments of the present invention may be in whole, or in part, replaced with such non-living makers, users, and/or participants with embodiments of the present invention. Likewise, when those skilled in the art identify such practical situations where such living makers, users, and/or participants with embodiments of the present invention may be in whole, or in part, replaced with such non-living makers, it will be readily apparent in light of the teachings of the present invention how to adapt the described embodiments to be suitable for such non-living makers, users, and/or participants with embodiments of the present invention. Thus, the invention is thus to also cover all such modifications, equivalents, and alternatives falling within the spirit and scope of such adaptations and modifications, at least in part, for such non-living entities.

Headings provided herein are for convenience and are not to be taken as limiting the disclosure in any way.

The enumerated listing of items does not imply that any or all of the items are mutually exclusive, unless expressly specified otherwise.

It is understood that the use of specific component, device and/or parameter names are for example only and not meant to imply any limitations on the invention. The invention may thus be implemented with different nomenclature/terminology utilized to describe the mechanisms/units/structures/components/devices/parameters herein, without limitation. Each term utilized herein is to be given its broadest interpretation given the context in which that term is utilized.

### Terminology

The following paragraphs provide definitions and/or context for terms found in this disclosure (including the appended claims):

“Comprising.” This term is open-ended. As used in the appended claims, this term does not foreclose additional structure or steps. Consider a claim that recites: “A memory controller comprising a system cache . . . .” Such a claim does not foreclose the memory controller from including additional components (e.g., a memory channel unit, a switch).

“Configured To.” Various units, circuits, or other components may be described or claimed as “configured to” perform a task or tasks. In such contexts, “configured to” or “operable for” is used to connote structure by indicating that the mechanisms/units/circuits/components include structure (e.g., circuitry and/or mechanisms) that performs the task or tasks during operation. As such, the mechanisms/unit/circuit/component can be said to be configured to (or be operable) for perform(ing) the task even when the specified mechanisms/unit/circuit/component is not currently operational (e.g., is not on). The mechanisms/units/circuits/components used with the “configured to” or “operable for” language include hardware—for example, mechanisms, structures, electronics, circuits, memory storing program instructions executable to implement the operation, etc. Reciting that a mechanism/unit/circuit/component is “configured to” or “operable for” perform(ing) one or more tasks is expressly intended not to invoke 35 U.S.C. sctn.112, sixth paragraph, for that mechanism/unit/circuit/component. “Configured to” may also include adapting a manufacturing process to fabricate devices or components that are adapted to implement or perform one or more tasks.

“Based On.” As used herein, this term is used to describe one or more factors that affect a determination. This term does not foreclose additional factors that may affect a determination. That is, a determination may be solely based on those factors or based, at least in part, on those factors. Consider the phrase “determine A based on B.” While B may be a factor that affects the determination of A, such a phrase does not foreclose the determination of A from also being based on C. In other instances, A may be determined based solely on B.

The terms “a”, “an” and “the” mean “one or more”, unless expressly specified otherwise.

Unless otherwise indicated, all numbers expressing conditions, concentrations, dimensions, and so forth used in the specification and claims are to be understood as being modified in all instances by the term “about.” Accordingly, unless indicated to the contrary, the numerical parameters set forth in the following specification and attached claims are approximations that may vary depending at least upon a specific analytical technique.

The term “comprising,” which is synonymous with “including,” “containing,” or “characterized by” is inclusive or open-ended and does not exclude additional, unrecited elements or method steps. “Comprising” is a term of art used in claim language which means that the named claim elements are essential, but other claim elements may be added and still form a construct within the scope of the claim.

As used herein, the phase “consisting of” excludes any element, step, or ingredient not specified in the claim. When the phrase “consists of” (or variations thereof) appears in a clause of the body of a claim, rather than immediately following the preamble, it limits only the element set forth in that clause; other elements are not excluded from the claim as a whole. As used herein, the phase “consisting essentially of” limits the scope of a claim to the specified elements or method steps, plus those that do not materially affect the basis and novel characteristic(s) of the claimed subject matter. Moreover, for any claim of the present invention which claims an embodiment “consisting essentially of” a certain set of elements of any herein described embodiment it shall be understood as obvious by those skilled in the art that the present invention also covers all possible varying scope variants of any described embodiment(s) that are each exclusively (i.e., “consisting essentially of”) functional subsets or functional combination thereof such that each of these plurality of exclusive varying scope variants each consists essentially of any functional subset(s) and/or functional combination(s) of any set of elements of any described embodiment(s) to the exclusion of any others not set forth therein. That is, it is contemplated that it will be obvious to those skilled how to create a multiplicity of alternate embodiments of the present invention that simply consisting essentially of a certain functional combination of elements of any described embodiment(s) to the exclusion of any others not set forth therein, and the invention thus covers all such exclusive embodiments as if they were each described herein.

With respect to the terms “comprising,” “consisting of” and “consisting essentially of” where one of these three terms is used herein, the presently disclosed and claimed subject matter may include the use of either of the other two terms. Thus in some embodiments not otherwise explicitly recited, any instance of “comprising” may be replaced by “consisting of” or, alternatively, by “consisting essentially of”, and thus, for the purposes of claim support and construction for “consisting of” format claims, such replacements operate to create yet other alternative embodiments “consisting essentially of” only the elements recited in the original “comprising” embodiment to the exclusion of all other elements.

Devices or system modules that are in at least general communication with each other need not be in continuous communication with each other, unless expressly specified otherwise. In addition, devices or system modules that are in at least general communication with each other may communicate directly or indirectly through one or more intermediaries.

A description of an embodiment with several components in communication with each other does not imply that all such components are required. On the contrary a variety of optional components are described to illustrate the wide variety of possible embodiments of the present invention.

As is well known to those skilled in the art many careful considerations and compromises typically must be made when designing for the optimal manufacture of a commercial implementation any system, and in particular, the embodiments of the present invention. A commercial implementation in accordance with the spirit and teachings of the present invention may configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

In the following description and claims, the terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements are in direct physical or electrical contact. However, “coupled” may also mean that two or more elements are not in direct contact with each other, but yet still cooperate or interact with each other.

It is to be understood that any exact measurements/dimensions or particular construction materials indicated herein are solely provided as examples of suitable configurations and are not intended to be limiting in any way. Depending on the needs of the particular application, those skilled in the art will readily recognize, in light of the following teachings, a multiplicity of suitable alternative implementation details.

Some embodiments of the present invention and variations thereof, relate to power tongue cleaners. In one embodiment of the invention, a power tongue cleaner may comprise of a detachable head and a base. The detachable head may further comprise of three cleaning blades.

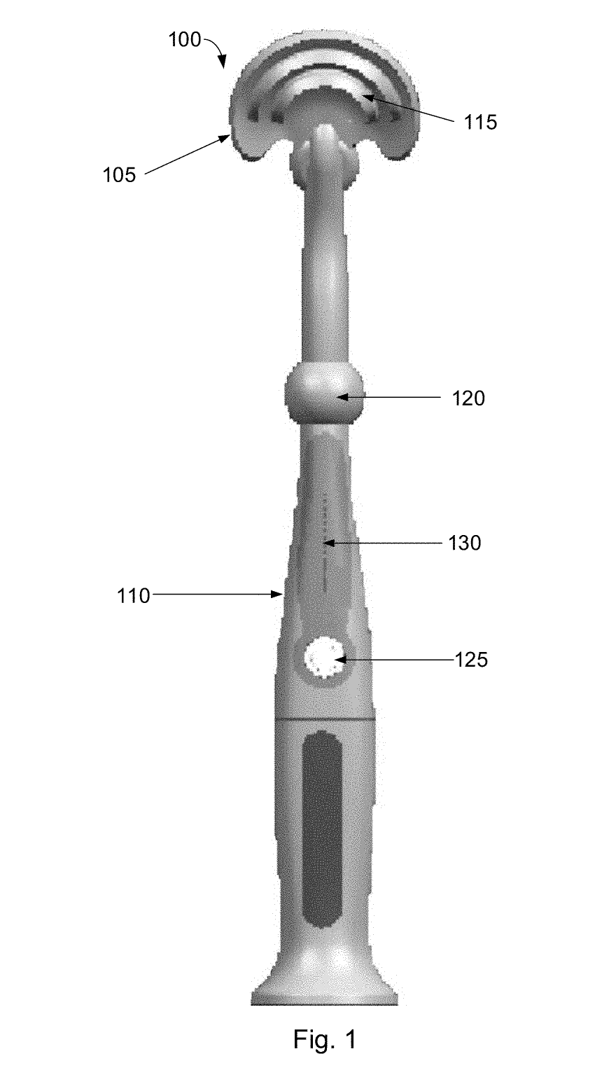

FIG. 1 illustrates an outside view of an exemplary power tongue cleaner 100, in accordance with an embodiment of the present invention. Power tongue cleaner 100 may comprise of a cleaning head 105 and a base 110. Cleaning head 105 may further comprise of three cleaning blades 115 and a ball base 120. Base 110 may further comprise of a power button 125 and an indicator 130. Virtually any material or materials may be used to construct cleaning head 105 and/or base 110. Materials may be selected for properties such as, without limitation, durability, flexibility, chemical stability, such as, but not limited to ABS plastic, PP plastic, other economical plastics compatible in quality and economical cost to ABS plastic, translucent and/or transparent of plastic, allowing the durability of the scraping edge to maintain sharpness for longer periods of time. In one embodiment of the preferred embodiment, power tongue cleaner 100 may be constructed from an acrylonitrile-butadiene-styrene (ABS) plastic for low cost production and Food and Drug Administration compliance.

The dimensions of cleaning head 105 may be designed such that a user's gag reflex is not triggered when using power tongue cleaner 100. Cleaning head 105 may have a spoon shape which may comprise of a concave indent in an oval shape. The three (3) scraping edges having the following specifications may be more effective in the scraping off of most, if not substantially all, typical decaying particles, including, but not limited to, bacterial thin films. As a non-limiting example of suitable design parameters suitable for many practical applications, the three scraping edges of the present embodiment may be constructed to have a width of the outer edge of 37 mm, middle edge of 27 mm, inside edge of 20 mm, and a distance between each edge of 5 mm. The three cleaning blades 115 may be designed to optimize the removal of bacteria, debris, residue, and/or other objects from the surface and/or deep crevices of a user's tongue. Cleaning blade design may include factors such as, without limitation, blade spacing, blade curvature, blade sharpness. It is contemplated that the blade sharpness of the 3 edges may be substantially identical A ball base 120 may be designed to allow for a user to maintain a better grip on power tongue cleaner 100 and/or allow for easier installing and/or removal of cleaning head 105.

Base 110 may be designed to accommodate virtually any electronics and/or power source. In one embodiment of the present invention, base 110 may be designed to internally hold all electronic components and a power source. A power button 125 may control the activation of a vibration function for power tongue cleaner 100, which may vibrate cleaning head 105 at a certain frequency, such as 30,000 strokes per minute. An indicator 130 may be virtually any indicator such as, without limitation, an LCD display, a series of LEDs, and a speaker. Indicator 130 may be used to indicate virtually any aspect of power tongue cleaner 100 such as, without limitation, power remaining, vibration speed, device status, etc. For example, without limitation, an on-off blue light may be employed to indicate whether the unit is turned on and off.

During typical operation, a user may grip base 110 and press power button 125. Once power button 125 is pressed, cleaning head 105 may vibrate and indicator 130 may display information to a user such as, without limitation, power remaining, vibration speed, device status. A user may insert power tongue cleaner 100 in the user's mouth and scrape the surface of the user's tongue with cleaning head 105. The three cleaning blades 115 may physically scrape away bacteria, residue, and/or matter. Vibration from cleaning head 105 may further assist a user in scraping away matter on the user's tongue by breaking up any matter on the user's tongue and/or freeing matter lodged in crevices in the user's tongue. Scraped and/or dislodged matter may be collected in between the three cleaning blades 115 and removed by the user after use.

It may be appreciated by a person with ordinary skill in the art, in light of and in accordance with the teachings of the present invention, that virtually any material and/or materials may be used to construct any part of power tongue cleaner 100. Materials may be, without limitation, copper, nickel, chrome, plastic, glass, etc. Depending upon the needs of the application, in some applications, steel may be too heavy and/or too expensive to use, and glass may be too dangerous to use as it may shatter inside mouth and cause damages. In another embodiment of the present invention, power tongue cleaner 100 may be made from stainless steel. In yet another embodiment of the present invention, cleaning head 105 may be constructed from an ABS plastic and base 110 may be constructed from aluminum.

It may be appreciated by a person with ordinary skill in the art, in light of and in accordance with the teachings of the present invention, that power tongue cleaner 100 may be integrated with virtually any oral hygiene too. Power tongue cleaner 100 may be designed with oral hygiene tools such as, without limitation, toothbrushes, floss picks, etc. In some embodiments, the power tongue cleaner's head may be replaced by a custom made toothbrush head that is configured to effectively operate at at suitable speed, such as, without limitation, 30000 strokes per minute. the heads can simply be replaced by each other.) In an alternative embodiment of the present invention, power tongue cleaner 100 may be combined with a toothbrush head and used as a dual purpose device.

It may be appreciated by a person with ordinary skill in the art, in light of and in accordance with the teachings of the present invention, ball base 120 may be designed in virtually any shape and/or size. Ball base 120 may be designed as, without limitation, a cone, a square, etc. The round shape may provide the safest way to make it stand alone and may provide the most comfortable hand grip to users. In an alternative embodiment of the present invention, ball base 120 may be a cone shape to guide a cleaning head 105 to a connection point on base 110 during installation.

It may be appreciated by a person with ordinary skill in the art, in light of and in accordance with the teachings of the present invention, that power tongue cleaner 100 may be constructed without electronics. In an alternative embodiment of the present invention, power tongue cleaner 100 may be constructed as a single piece without a vibration function.

It may be appreciated by a person with ordinary skill in the art, in light of and in accordance with the teachings of the present invention, that base 110 may be designed in virtually any shape and/or size. Base 110 may be designed according to factors such as, without limitation, a specific user, a specific mounting and/or standing position, accommodating internal components, etc. It is contemplated that in many practical embodiments the round shape may provide a safer way to make it stand alone and may provide a more comfortable hand grip to users. In some embodiments, The base design may also include a rubber thumb guide to provide a more non-slip and more firm grip capability. the thickness may be designed to provide comfort with smaller hands. In another embodiment of the present invention, base 110 may be constructed in an ergonomic shape for a user to hold.

It may be appreciated by a person with ordinary skill in the art, in light of and in accordance with the teachings of the present invention, base 110 may be designed to mount to any surface. Base 110 may be designed with virtually any mounting and/or standing means such as, without limitation, magnets, base stands, adhesives, hooks, etc. In an alternative embodiment of the present invention, base 110 may be designed with suction cups for wall mounting.

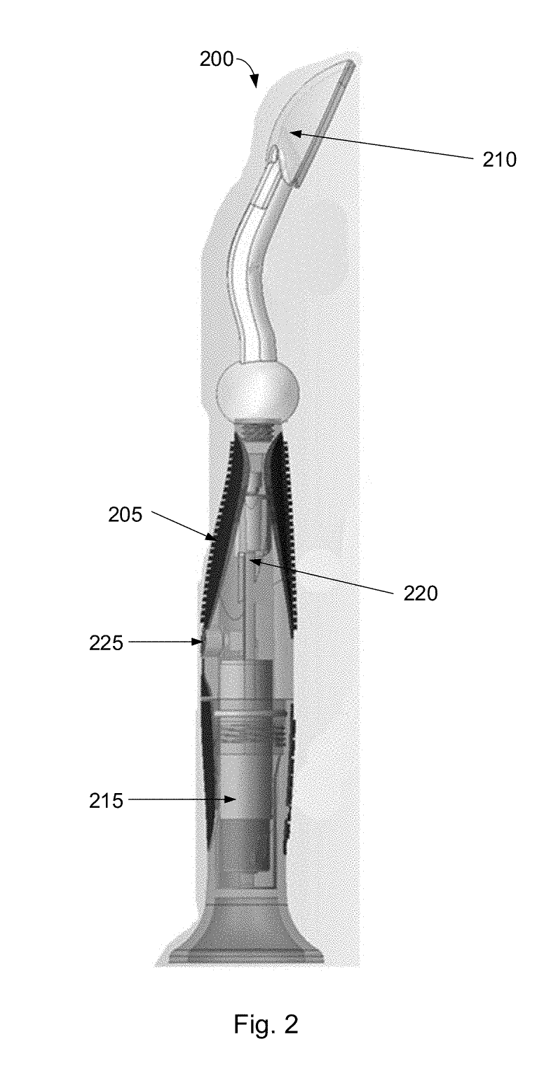

FIG. 2 illustrates an internal view of an exemplary power tongue cleaner 200, in accordance with an embodiment of the present invention. Power tongue cleaner 200 may comprise of a base 205 and a cleaning head 210. Base 205 may further comprise of a battery 215, a motor 220, and a power button 225. Motor 220 may be controlled by a user with power button 225 and draw power from battery 215. Cleaning head 210 may couple with base 205 by virtually any means such as, without limitation, interlocking joints, threaded screws. In order to provide a firm and safe attachment, a preferred embodiment would use a push in turn to lock system allowing for a firmer and longer lasting attachment.

During typical operation, a user may press power button 225 to send power from battery 215 to motor 220. Motor 220 may operate on a gear set and/or directly move cleaning head 210. An on and off light allows a user to be able to determine how long it should be used as they scrape off all areas of mouth. Movement in cleaning head 210 may vibrate cleaning blades located on cleaning head 210 which may break up matter located on a user's tongue. The cleaning blades on cleaning head 210 may vibrate in virtually any direction and/or directions such as, without limitation, horizontally, vertically, up and down. The power tongue cleaner of the present embodiment may employ provide omnidirectional vibrations as a user moves up and down to scrape off decaying particles. The powerful vibration will orchestrate to effectively scrape all tissues of the tongue within a short period of time effectively. The vibration frequency of the cleaning blades in cleaning head 210 may be any suitable frequency such as, without limitation, 20,000 to 35,000 rpms. It is contemplated that in many practical embodiments a frequency of 30,000 rpms may be more effective over most, if not all, possible durations as a user may have different time availability.

It may be appreciated by a person with ordinary skill in the art, in light of and in accordance with the teachings of the present invention, that base 205 may not necessary have to contain part or all of its components within base 205's enclosure. For embodiments where waterproofing is not so important, the components may be located outside and/or as an extension of base 205. It is contemplated that in many practical embodiments the waterproofing will be of some importance to electronic components such as, but not limited to, a battery, motor, light bulb, wiring, etc., and it may be desirable to locate such sensitive components inside the handle. In alternative embodiment of the present invention, battery 215 and motor 220 may be modularly attached to one end of base 205.

It may be appreciated by a person with ordinary skill in the art, in light of and in accordance with the teachings of the present invention, that virtually any power source may be implemented in place of battery 215. Power sources may be, without limitation, compress gas, wind-up mechanisms, gyroscopic mechanisms. It is contemplated that in many practical embodiments unit should preferably be light enough for a user to hold and effectively move up/down, the best power being a AAA battery. In an alternative embodiment of the present invention, battery 215 may be a wind-up mechanism that stores kinetic energy.

It may be appreciated by a person with ordinary skill in the art, in light of and in accordance with the teachings of the present invention, that cleaning head 210 may be omnidirectional. as a user will move the power tongue cleaner in a horizontal, vertical and up and down motion. In an alternative embodiment of the present invention, cleaning head 210 may alternatingly vibrate horizontally and vertically to better remove matter off a user's tongue. One aspect of the present embodiment is to enable bringing residue to the surface from between taste buds and loosening it from the tongue and effectively removing the residue. It may be appreciated by a person with ordinary skill in the art, in light of and in accordance with the teachings of the present invention, that cleaning head 210 may vibrate at virtually any non-uniform frequencies and/or intensities. In an alternative embodiment of the present invention, cleaning head 210 may modulate between a frequency range to better scrape matter off a user's tongue with a 30,000 rpm vibration as often being a frequency that provides a higher level of strength in many practical applications.

It may be appreciated by a person with ordinary skill in the art, in light of and in accordance with the teachings of the present invention, that power tongue cleaner 200 may vibrate cleaning head 205 according to and algorithm and/or feedback loop. A processing unit and/or hardware loop may be designed to execute and algorithm and/or feedback loop in response to virtually any factor such as, without limitation, remaining device power, cleaning head resistance, user input, etc. In an alternative embodiment of the present invention, power tongue cleaner 200 may implement a hardware feedback loop that increases vibrational intensity if cleaning head 205 meets any physical resistance.

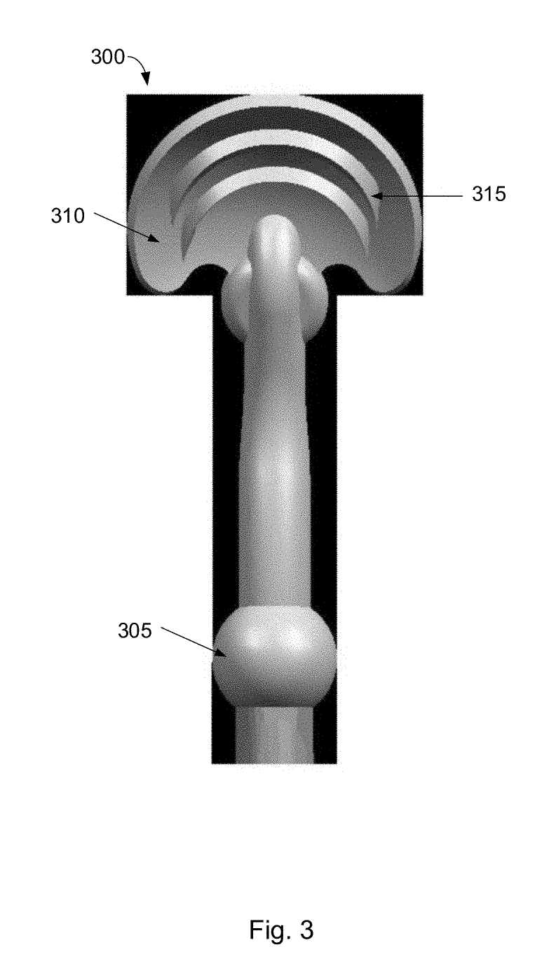

FIG. 3 illustrates an outside view of an exemplary cleaning head 300, in accordance with an embodiment of the present invention. Cleaning head 300 may comprise of a connection point 305, a head section 310, and cleaning blades 315. Connection point 305 is a coupling point between cleaning head 300 and the base portion of a power tongue cleaner. Connection point 305 may be designed in virtually any shape and size. with a preferred round embodiment. as the power tongue cleaner has a ball to stop slipping of a user's hand and also provides a gliding smooth surface, ensuring safe surface to mouth. Head section 310 may be designed such that a user's gag reflex is reduced when inserting cleaning head 300 into their mouth. Designs for head section 310 may be virtually any design such as, without limitation, a spoon shape, concaved, etc. A user is used to a spoon shape for many years and gagging reflexes of this shape have been overcome. Other shapes, not familiar to the mouth, may automatically trigger gagging reflexes. The spoon shape somewhat follows the design of the mouth to allow the power tongue cleaner to reach further back where the majority of harmful bacteria and food particles hide, without triggering the gag reflex, which may be triggered by the touching the uvula. Cleaning blades 315 may be one or more blades of virtually any design. Cleaning blades 315 may be three blades designed, without limitation, curvatures, staggered heights, etc. In order to follow contours of tongue muscles effectively, it should be round as the power tongue cleaner. scrapes, collects and removes particles effectively out of mouth. The size and shape of the power tongue cleaner represents the optimum shape and size. Any matter scraped by cleaning blades 315 may collect in between the cleaning blades 315 and/or head section 310's outer edge and be removed by the user after cleaning.

It may be appreciated by a person with ordinary skill in the art, in light of and in accordance with the teachings of the present invention, that virtually any number of cleaning blades 315 may be designed into cleaning head 300. Factors that determine a number of cleaning blades 315 to be designed into cleaning head 300 may be, without limitation, cost, cleaning effectiveness, durability, etc.

In some embodiments, the motor may provide 30,000 revolutions per minute, and in order to scrape off all possible particles, one or two cleaning blades will not be enough to scrape and remove the particles. Three blades are designed to provide an effective scraping and removing action, all at the same time. Based on the sizes and distances between blades, separated by 5 mm, inside the head, 2 or 4 blades may not be as effective as the 3 blades in many practical applications. Three blades is often more optimal at least because in many practical applications there needs to be enough room in the head to collect the debris. Four blades typically may not allow for enough room to collect debris between the blades. 3 blades with a 5 mm distance between each other, is contemplated to be the more effective in many practical applications to remove undesirable particles in a single stroke as opposed to one or two blades. In another embodiment of the present invention, cleaning head 300 is designed with three cleaning blades 315 to effectively remove matter from a user's tongue.

It may be appreciated by a person with ordinary skill in the art, in light of and in accordance with the teachings of the present invention, that cleaning blades 315 may be modular and/or operate independently from each other. Cleaning blades 315 may be, without limitation, heterogeneous blades of different designs, vibrate at different frequencies and/or intensities, created from different materials, etc. Particles scraped off by all three blades will be removed outside of the mouth by the outer blade, the middle blade is smaller than the outer blade and the inside blade is smaller than the middle blade. In an alternative embodiment of the present invention, cleaning blades 315 may be modularly replaced and be of varying sharpness to more effectively remove matter from a user's tongue.

It may be appreciated by a person with ordinary skill in the art, in light of and in accordance with the teachings of the present invention, that cleaning blades 315 may be made from virtually any material and/or compromise of a plurality of pieces. Cleaning blades 315 may be made from materials such as, without limitation, sponge, cloth, etc. with a preferred embodiment using ABS solid plastic in order to scrape off and remove all possible particles. Porous materials will not be as effective, because small scraped off particles, especially harmful bacteria will get into porous materials and will not be effectively removed. In an alternative embodiment of the present invention, cleaning blades 315 may be made from tightly packed brush bristles to better remove matter in a user tongue's crevices.

It may be appreciated by a person with ordinary skill in the art, in light of and in accordance with the teachings of the present invention, that cleaning head 300 may include virtually any additional oral cleaning technology. Additional oral cleaning technologies may include without limitation, an anti-bacterial coating, ultrasounds, etc. In another embodiment of the present invention, cleaning head 300 may additionally comprise of an ultrasound mechanism that may break down matter on a user's tongue during cleaning. In an alternative embodiment of the present invention, cleaning head 300 may additionally comprise of an ultraviolet light that may dissolve bacteria on a user's tongue during cleaning.

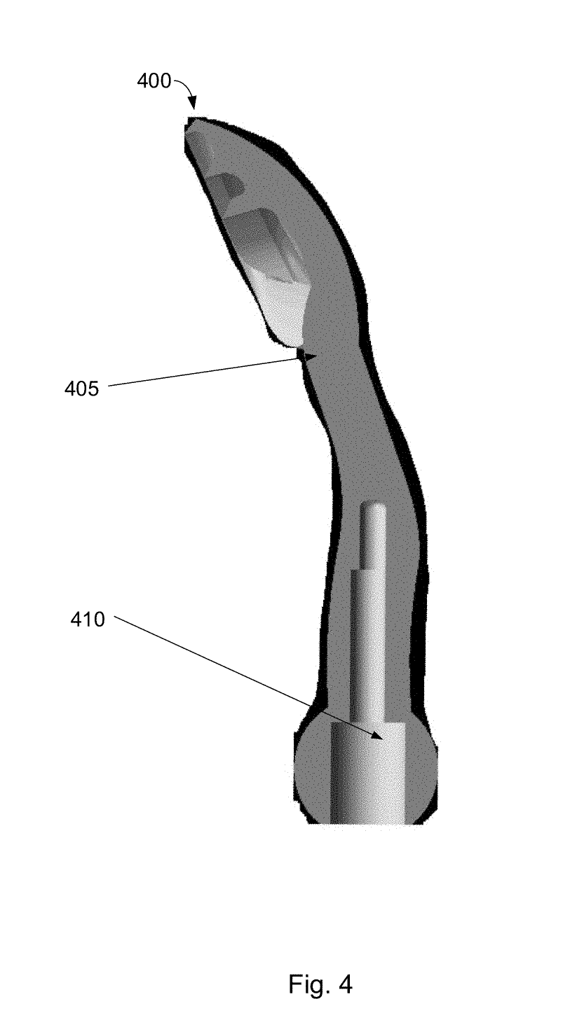

FIG. 4 illustrates an internal view of an exemplary cleaning head 400, in accordance with an embodiment of the present invention. Cleaning head 400 may comprise of a cleaning head section 405 and a vibrational mechanism 410. Cleaning head section 405 vibrates according to vibrational mechanism 410 which may be virtually any gearing and/or vibrating mechanism such as, without limitation, a linear actuator, a moving weight, etc. In many practical applications, in order to provide omnidirectional vibrations, the motor is better located inside of the handle as often direct vibration of head may cause users to lose control of scraping head and could damage tongue tissues. Vibrational mechanism 410 may vibrate cleaning head 400 in virtually any direction and/or directions such as, without limitation, horizontally, vertically, up and down, with omnidirectional vibrations inside the handle being a more desired vibration for the head blades.)

It may be appreciated by a person with ordinary skill in the art, in light of and in accordance with the teachings of the present invention, cleaning head section 405 may comprise of multiple interconnected segments. In an alternative embodiment of the present invention, cleaning head section 405 may comprise of three interconnected segments that allow for multiple degrees of freedom between each segment. In many practical applications The handle with 30,000 rpms represents an optimal vibration for the power tongue cleaner. It should be noted that rotating blades in many practical applications could be dangerous to soft tongue tissues.

It may be appreciated by a person with ordinary skill in the art, in light of and in accordance with the teachings of the present invention, that vibrational mechanism 410 may simply be a focus point for movement from a motor in the base portion of a power tongue cleaner. In an alternative embodiment of the present invention, cleaning head 400 may be a single piece that is vibrated by a power tongue cleaner's base section.

In some embodiments of the present invention the power tongue cleaner, between the scraping head and ball, may be ergonomically designed in such a way to allow the user to remove debris from the tongue more comfortably.

In some embodiments of the present invention a rubber o-ring may be suitably configured into the bottom of the base where it connects to the top of the base to replace battery, which may better help keep the area sealed and resist water to penetrate the powered tongue cleaner.

In some embodiments of the present invention when the power tongue cleaner stands on its own, the head may be configured to be angled down to assist in drying, which may be important in some situations where there is concern that moisture may breed harmful bacteria.

In some embodiments of the present invention, the bottom of the base may be designed to include suction cup.

It is contemplated that in many practical embodiments the 3 scraping edge design may put less pressure per blade on the tongue while scraping, thereby potentially providing a safer scraping experience.

All the features disclosed in this specification, including any accompanying abstract and drawings, may be replaced by alternative features serving the same, equivalent or similar purpose, unless expressly stated otherwise. Thus, unless expressly stated otherwise, each feature disclosed is one example only of a generic series of equivalent or similar features.

It is noted that according to USA law 35 USC §112 (1), all claims must be supported by sufficient disclosure in the present patent specification, and any material known to those skilled in the art need not be explicitly disclosed. However, 35 USC §112 (6) requires that structures corresponding to functional limitations interpreted under 35 USC §112 (6) must be explicitly disclosed in the patent specification. Moreover, the USPTO's Examination policy of initially treating and searching prior art under the broadest interpretation of a “mean for” claim limitation implies that the broadest initial search on 112(6) functional limitation would have to be conducted to support a legally valid Examination on that USPTO policy for broadest interpretation of “mean for” claims. Accordingly, the USPTO will have discovered a multiplicity of prior art documents including disclosure of specific structures and elements which are suitable to act as corresponding structures to satisfy all functional limitations in the below claims that are interpreted under 35 USC §112 (6) when such corresponding structures are not explicitly disclosed in the foregoing patent specification. Therefore, for any invention element(s)/structure(s) corresponding to functional claim limitation(s), in the below claims interpreted under 35 USC §112 (6), which is/are not explicitly disclosed in the foregoing patent specification, yet do exist in the patent and/or non-patent documents found during the course of USPTO searching, Applicant(s) incorporate all such functionally corresponding structures and related enabling material herein by reference for the purpose of providing explicit structures that implement the functional means claimed. Applicant(s) request(s) that fact finders during any claims construction proceedings and/or examination of patent allowability properly identify and incorporate only the portions of each of these documents discovered during the broadest interpretation search of 35 USC §112 (6) limitation, which exist in at least one of the patent and/or non-patent documents found during the course of normal USPTO searching and or supplied to the USPTO during prosecution. Applicant(s) also incorporate by reference the bibliographic citation information to identify all such documents comprising functionally corresponding structures and related enabling material as listed in any PTO Form-892 or likewise any information disclosure statements (IDS) entered into the present patent application by the USPTO or Applicant(s) or any 3^{rd} parties. Applicant(s) also reserve its right to later amend the present application to explicitly include citations to such documents and/or explicitly include the functionally corresponding structures which were incorporate by reference above.

Thus, for any invention element(s)/structure(s) corresponding to functional claim limitation(s), in the below claims, that are interpreted under 35 USC §112 (6), which is/are not explicitly disclosed in the foregoing patent specification, Applicant(s) have explicitly prescribed which documents and material to include the otherwise missing disclosure, and have prescribed exactly which portions of such patent and/or non-patent documents should be incorporated by such reference for the purpose of satisfying the disclosure requirements of 35 USC §112 (6). Applicant(s) note that all the identified documents above which are incorporated by reference to satisfy 35 USC §112 (6) necessarily have a filing and/or publication date prior to that of the instant application, and thus are valid prior documents to incorporated by reference in the instant application.

Having fully described at least one embodiment of the present invention, other equivalent or alternative methods of implementing power tongue cleaners according to the present invention will be apparent to those skilled in the art. Various aspects of the invention have been described above by way of illustration, and the specific embodiments disclosed are not intended to limit the invention to the particular forms disclosed. The particular implementation of the power tongue cleaners may vary depending upon the particular context or application. By way of example, and not limitation, the power tongue cleaners described in the foregoing were principally directed to oral hygiene tool implementations; however, similar techniques may instead be applied to cleaning tools, surface cleaners, or vehicle windshield wipers, which implementations of the present invention are contemplated as within the scope of the present invention. The invention is thus to cover all modifications, equivalents, and alternatives falling within the spirit and scope of the following claims. It is to be further understood that not all of the disclosed embodiments in the foregoing specification will necessarily satisfy or achieve each of the objects, advantages, or improvements described in the foregoing specification.

Claim elements and steps herein may have been numbered and/or lettered solely as an aid in readability and understanding. Any such numbering and lettering in itself is not intended to and should not be taken to indicate the ordering of elements and/or steps in the claims.

The corresponding structures, materials, acts, and equivalents of all means or step plus function elements in the claims below are intended to include any structure, material, or act for performing the function in combination with other claimed elements as specifically claimed.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The Abstract is provided to comply with 37 C.F.R. Section 1.72(b) requiring an abstract that will allow the reader to ascertain the nature and gist of the technical disclosure. It is submitted with the understanding that it will not be used to limit or interpret the scope or meaning of the claims. The following claims are hereby incorporated into the detailed description, with each claim standing on its own as a separate embodiment.

What is claimed is:

1. A device comprising:
a cleaning head component, said cleaning head component is a removable and replaceable cleaning head component configured to clean a surface of a tongue, said cleaning head component comprising:
a head section, wherein said head section comprises a spoon shaped head section configured to be operable to reduce a user's gag reflex;
a cleaning blade segment, wherein said cleaning blade segment comprises three blades configured to form a triple edged blade scraping segment operable to remove at least one of a bad breath causing bacteria, a bacterial biofilms, a decaying food particle, a fungi, and a dead cell lodged in a crevice of said tongue surface;
a ball base segment, said ball base segment is configured to allow a user to maintain a grip on said device and allow for installation and removal of said cleaning head component; and
a vibrational mechanism, said vibrational mechanism is configured to vibrate said cleaning head in a predetermined direction of motion;
and
a base component configured to house electronic components, said base component is further configured attach to said cleaning head component, said base component comprising;
a power source;
a motor configured to vibrate said cleaning head component at a predetermined rate of said direction of motion, wherein said motor draws power from said power source;
a power button configured to activate and deactivate said motor, thereby, control the activation of said cleaning head vibration;
an indicator, wherein said indicator comprises an On-Off blue light that is configured to indicate if the device is turned on or off.

2. The device of claim 1, wherein said ball base segment comprises a first connection point for connection to said base component, and wherein said base component comprises a second connection point for connection to said first connection point configured to form a Push in-Turn to Lock configuration for secure attachment of said cleaning head component with said base component.

3. The device of claim 1, wherein said vibrational mechanism is configured to vibrate said cleaning head component in a proximate up-and-down motion, in a proximate vertical direction, and in which said indicator further comprising a display configured to display a vibration speed.

4. The device of claim 1, in which each of said three blades forming said triple edged blade scraping segment is individually replaceable.

5. A device comprising:
a cleaning head, said cleaning head is a removable and replaceable cleaning head for cleaning a surface, said cleaning head comprising:
a head section, wherein said head section comprises a spoon shaped head section, said spoon shaped head section is configured to be operable to reduce a user's gag reflex;
a cleaning blade segment, wherein said cleaning blade segment comprises three blades configured to form a triple edged scraping implement; and
a ball base segment, said ball base segment is configured to aid a user in the installation and removal of said cleaning head component;
and
a base component configured to attach to said cleaning head component, said base component comprising;
a power source for providing power;
a motor configured to vibrate said cleaning head component at a predetermined direction of motion, wherein said motor draws power from said power source;
a power button configured to activate or deactivate said motor; and
an indicator configured to indicate a status of said device.

6. The device of claim 5, wherein said ball base segment comprises a first connection point for connection to said base component.

7. The device of claim 6, wherein said base component comprises a second connection point for connection to said first connection point.

8. The device of claim 7, in which said first connection point and second connection point comprising a Push in Turn to Lock system configured for secure attachment of said cleaning head component with said base component.

9. The device of claim 5, wherein said indicator comprising an On-Off blue light configured to indicate if the device is turned on or off.

10. The device of claim 5, wherein said base component further comprising a removable suction cup for wall mounting.

11. The device of claim 5, wherein said motor is a 30,000 RPM motor.

12. The device of claim 5, wherein each of said three blades forming said triple edged scraping implement is individually replaceable.

13. The device of claim 5, in which said indicator further comprising a display configured to display an amount of remaining power.

14. The device of claim 5, in which said indicator further comprising a display configured to display a vibration speed.

15. The device of claim 5, wherein said cleaning head component is a removable toothbrush and wherein said head section is a toothbrush section.

16. The device of claim 5, in which said cleaning head component further comprising a vibrational mechanism configured to vibrate said cleaning head component when said power button activates said motor.

17. The device of claim 16, in which said vibrational mechanism is configured to vibrate said cleaning head component in a proximate up-and-down motion, in a proximate vertical direction.

18. The device of claim 17, in which said vibrational mechanism is configured to vibrate said cleaning head component in a proximate up-and-down motion, in a proximate horizontal direction.

19. A device comprising:
means for cleaning comprising:
means for reducing a user's gag reflex, wherein said reducing means comprises a spoon shaped reducing means;
means for forming a triple edged scraping head operable to clean a surface of a tongue; and
means for aiding an installation and removal of said cleaning means;
and
means for housing electronic components, said housing means is configured to attach to said cleaning means, said housing means comprising;
means for providing power;
means for vibrating said cleaning means;
means for controlling the activation of said vibrating means; and
means for indicating a status of said cleaning means.

20. The device of claim 19, in which said installation aiding means comprises means for connection in a Push in Turn to Lock attachment to said housing means.
